(19) United States
(12) Patent Application Publication
(10) Pub. No.: US 2010/0249607 A1
(43) Pub. Date: Sep. 30, 2010

(54) QUANTITATIVE SPECTROSCOPIC IMAGING

(75) Inventors: Chung-Chieh Yu, Tuscon, AZ (US); Condon Lau, Cambridge, MA (US); Ramachandra Dasari, Shererville, IN (US); Michael Feld, Jamaica Plain, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: MASSACHUSETTS INSTITUTE OF TECHNOLOGY, Cambridge, MA (US)

(21) Appl. No.: 12/568,459

(22) Filed: Sep. 28, 2009

Related U.S. Application Data
(60) Provisional application No. 61/194,457, filed on Sep. 26, 2008.

(51) Int. Cl. A61B 6/00; A61B 1/06

(52) U.S. Cl. 600/476; 600/182

(57) ABSTRACT

The present invention relates to a fully quantitative spectroscopy imaging instrument for wide area detection of early cancer (dysplasia). This instrument provides quantitative maps of tissue biochemistry and morphology, making it a powerful surveillance tool for objective early cancer detection. The design, construction, calibration, and diagnostics applications of this system is described with the use of physical tissue models. Measurements were conducted on a resected colon adenoma, and the system can be used for vivo imaging in the oral cavity.

### CROSS REFERENCE TO RELATED APPLICATION

This application claims priority to U.S. Application 61/194,457 filed on Sep. 26, 2008 the entire contents of which are incorporated herein by reference.

### BACKGROUND OF THE INVENTION

Early detection is essential for managing cancer, since treatment is much more successful when lesions are diagnosed at an early, noninvasive stage. Current cancer diagnosis often employs visual inspection of a wide area of tissue followed by biopsy of suspicious sites. This practice is problematic for two reasons: (1) early cancers are not always detectable by visual inspection, so, unavoidably, unnecessary biopsies are taken for precautionary reasons and invisible lesions are missed; and (2) biopsy suffers from undersampling, the results are subjective, and the resulting pathology can be subject to low inter-observer agreement. Furthermore, biopsy results often are not available immediately, resulting in delayed treatment and patient anxiety. Much attention has been focused on spectroscopy, particularly reflectance and fluorescence spectroscopy, to overcome these problems. Reflectance and fluorescence are known to exhibit spectral features associated with the different morphology and biochemistry of normal and cancerous tissues. These techniques have the capability to detect invisible lesions, and to provide quantitative diagnostic information for objective evaluation.

However, there is a continuing need for improvements in spectroscopic techniques for the measurement and diagnosis of tissue.

### SUMMARY OF THE INVENTION

The present invention relates to systems and methods using reflectance and fluorescence for imaging tissue for spectroscopic diagnosis. Preferred embodiments utilize an optical fiber probe for light delivery and collection for a variety of applications including in the cervix, oral cavity, esophagus, colon, lung, and bladder, with various degrees of quantitative analysis. Specifically, the present invention uses quantitative methods for tissue diagnosis in which diffuse reflectance and fluorescence, in combination, are use to extract quantitative information about morphological and biochemical tissue constituents.

The present invention uses systems and methods quantitative spectroscopy (QS). Diffuse reflectance spectra from tissue are analyzed using a representation or model to obtain information about hemoglobin concentration and saturation, light scattering parameters, and other tissue characteristics. This method is known as diffuse reflectance spectroscopy (DRS). Tissue fluorescence, collected from the same spot at the same time, is analyzed using the diffusely reflected light to remove spectral distortions, resulting in the “intrinsic fluorescence” that can be observed in the absence of scattering and absorption, from which contributions from tissue fluorophores can then be extracted. This method is known as intrinsic fluorescence spectroscopy (IFS). Histological parameters are then extracted by fitting the observed spectra to parameters such as tissue density, blood concentration and oxygenation, and concentrations of collagen and reduced nicotinamide adenine dinucleotide (NADH), determined from calibration of physical models of tissue with known features.

Contact probe techniques are promising, but like biopsy, suffer from undersampling. To overcome this, wide area light collection and imaging in fluorescence and reflectance tissue diagnosis, is used to provide quantitative analysis. A preferred embodiment uses a model-based, quantitative approach to wide field imaging that is referred to herein as quantitative spectroscopic imaging (QSI). Data are collected by means of a non-contact “virtual” probe, imaged at the tissue surface. This virtual probe is then raster scanned to interrogate a wide tissue area (for example, in a range of 1-4 cm²), using one or more spots (1 mm²) at a time. The quantitative measurements of tissue properties enable the spectra for each pixel to be analyzed using probe methodology. Hence, the QSI images are directly interpretable in terms of histological features, thus providing an accurate diagnosis.

A preferred embodiment of the present invention involves the design, construction, calibration, and the clinical application of this QSI system. Measurements using physical tissue models (“phantoms”) demonstrate the accuracy of QSI. Ex vivo spectral images of a resected colon adenoma demonstrate its ability to diagnose and image malignant lesions. In addition, in vivo spectral images from a hyperkeratotic lesion on the ventral surface of the tongue further demonstrates clinical applicability.

QSI system is preferably used for imaging early stage cancer. The present invention provides for quantitative mapping methodology. This instrument is the first that provides quantitative maps of tissue biochemistry and morphology for wide area cancer imaging.

### DESCRIPTION OF THE DRAWINGS

FIGS. 1A-1C include illustrations of a contact probe and a non contact probe where the central beam is for illumination and outer beams are for collection, in which a scanning spot of light is delivered to the tissue surface, the spot preferably being about the diameter of an optical fiber contact probe, a schematic diagram of the QSI instrument and a schematic diagram of the optical head, respectively.

FIG. 2. is calibrated reflectance spectra (solid lines) measured from the same imaging position on different tissue phantoms (90, 92, 94) with the best fit spectra using the approach of Zonios et al. are also plotted (dashed lines), the characteristic absorption bands of hemoglobin at 420 nm, 540 nm, and 580 nm are visible.

FIG. 3 shows bulk fluorescence spectra measured from the same imaging position on different tissue phantoms 97, 96, and 98 (as indicated in Table 2), the corresponding intrinsic fluorescence spectra 95 extracted using the method of Müller et al. are also plotted (IFS) where the dashed spectrum is the fluorescence measured from furan in water without intralipid or hemoglobin present. It is nearly identical to the dashed lines. Note that the IFS spectra and the spectrum of furan in water all overlap.

FIGS. 4A-4F show white light image of ex vivo colon specimen. FIG. 4B: Ex vivo colon pathology, the specimen is sectioned from the region indicated by the left line (A-B) in FIG. 4B. Parameter maps of A, c_{Hb}, and c_{Coll}/c_{NADH} are shown in FIGS. 4C-4E and maps can be compared to the picture in FIG. 4A by matching the coordinates with units in millimeters; FIG. 4F: Diagnosis map with black normal and white cancerous tissue, the diagnosis using QSI is constant to the diagnosis using histopathology which indicates the area below line 100 in FIG. 4A is cancerous.

FIG. 5A-5D show a white light image of ventral tongue site with the boundaries, between hyperkeratosis and normal areas, delineated by the dashed yellow lines. The coordinates are in millimeters and the data in FIGS. 5B-5D can be compared to the picture in FIG. 5A by matching the coordinates, namely, A parameter (FIG. 5B), α parameter (FIG. 5C), c_{Coll}/c_{NADH} parameter (FIG. 5D).

FIG. 6 is a perspective view of the optical head module.

FIG. 7 illustrates the use of a scanning light region or spot using a fiber optic probe.

FIGS. 8A-8B illustrate a light delivery and collection imaging probe.

FIG. 9 graphically illustrates reflectance spectra from free space and endoscope QSI system.

FIG. 10 illustrates the mobile module connected to the endoscopic imaging system.

### DETAILED DESCRIPTION OF THE INVENTION

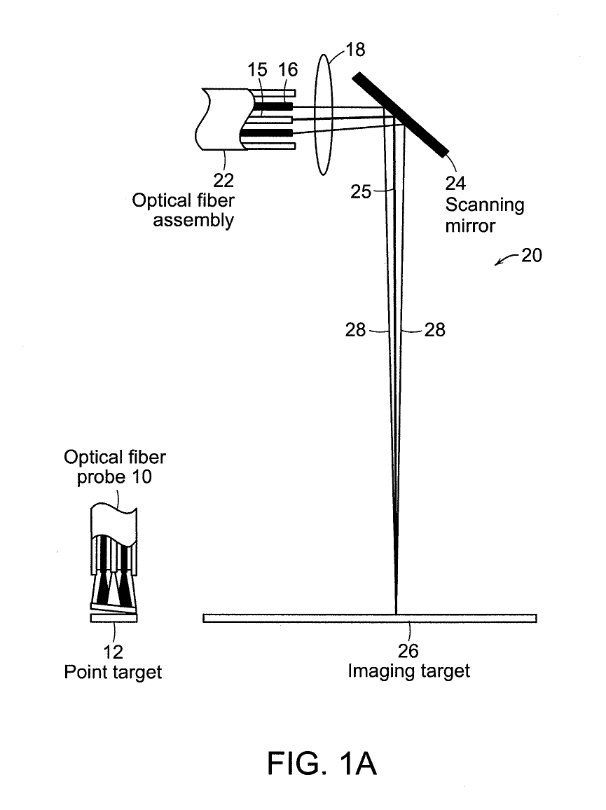

The present invention relates to the use of a scanning light region to quantitatively measure objects at a distance. Instead of the quantitative methodology of contact probes, such as those described by: J. W. Tunnell et al., “Instrumentation for multi-modal spectroscopic diagnosis of epithelial dysplasia,” Technol. Cancer Res. Treat. 2, 505-514 (2003), the entire contents of which is incorporated herein by reference, the present invention provides a wide area imaging instrument. In the contact probe 10 geometry, such as seen in FIG. 1A where the optical fiber probe 10 consists of a single light delivery fiber surrounded by six collection fibers. All fibers are fused together at the tip to form an optical shield approximately 1 mm long. This arrangement of fibers and quartz shield shaped at a 17 degree angle at the tip provides a reproducible geometry of overlapping excitation and collection cones, creating a fixed distance between fiber tips and tissue and a sampling spot 12 on the tissue surface with a diameter of approximately 800 micrometer. In the imaging implementation, an optical fiber assembly 22 provides illuminating light 25 that is relayed by optics 18 onto a small region of the tissue, providing a virtual non-contact probe. Wide area coverage is achieved by scanning the imaged spot over the tissue 26 with a scanner 24. Note that in this case the arrangement of the delivery and collection fibers 15, 16 can be precisely controlled, providing increased optical design flexibility. Light is collected along return paths 28 with scanner 24 onto collection fibers 16.

The characteristics of a non-contact probe (spot size˜1 mm in diameter and NA˜0.02) differ somewhat from that of contact probe system (spot size˜0.8 mm in diameter and NA˜0.22). As discussed below, the probe parameters are incorporated in a reflectance measurement, such as those described by: G. Zonios et al., “Diffuse reflectance spectroscopy of human adenomatous colon polyps in vivo,” Appl. Opt. 38, 6628-6637 (1999) and using fluorescence as described by: M. G. Muller et al., “Intrinsic fluorescence spectroscopy in turbid media: disentangling effects of scattering and absorption,” Appl. Opt. 40, 4633-4646 (2001), both these publications being incorporated herein by reference, where differences in probe geometries can be taken into account, so the correct tissue parameters can be obtained from measurements with either probe. This robustness is an important virtue of the present quantitative approach.

The QSI instrument has been developed for clinical settings, and must be portable. As shown in FIGS. 1B and 1C, portability is achieved by placing the heaviest components in a mobile cart 40 and creating a separate lightweight, mobile optical head 58 to be operated by the user, such as a physician. The design is modular where three larger modules, mounted in a cart, are connected to the optical head by means of optical fibers 54, 56. Module 1, the light source module 46, such as a 75 W CW white light arc lamp 50 (Simplicity series, Newport Corp.) and a nitrogen laser 52 that delivers 337 nm light pulses of duration <3.5 ns and energy 175 μJ at 20 Hz (NL100, Stanford Research Systems Inc.). Module 2 contains a spectrograph/CCD light detecting system 44 (Princeton Instruments Corp.). Module 3 is the computer 42, which controls data acquisition and analyzes the data. National Instruments Labview software and DAQ data acquisition hardware are used to control and coordinate the various components. Module 4, the light delivery and collection system 58 is mounted on an articulated arm for easy maneuvering. It can be located on a smaller cart with wheels, so that the larger equipment cart can be located away from the patient, since space in hospital procedure rooms is limited. Additional systems and methods are described in U.S. application Ser. No. 11/492,298 filed on Jul. 25, 2006, also incorporated herein by reference in its entirety.

FIG. 1C is a schematic diagram of the optical head 58. White light (with NA˜0.02) from the xenon arc lamp 50 (CW) illuminates a 1 mm diameter “diagnostic spot” or region on the tissue at a distance 76 about 20-25 cm away from the optical head. Diffusely reflected light from a 2 mm diameter circle centered on the diagnostic spot is relayed back to the optical head and focused onto eight collection fibers 75 with aperture 70 and coupled to a spectrograph and CCD. Each of the eight fibers collects light returning from the tissue in different directions, which helps avoid specular reflection. Next, 337 nm light (with NA˜0.02) from the nitrogen laser 52 illuminates the same diagnostic spot through fiber coupler 48, fibers 56, lenses 67,69, rod mirror 66 and dichroic mirror 68, and fluorescence is collected in the same manner as diffuse reflectance. These two measurements serve to provide the reflectance and fluorescence measurements of a contact probe.

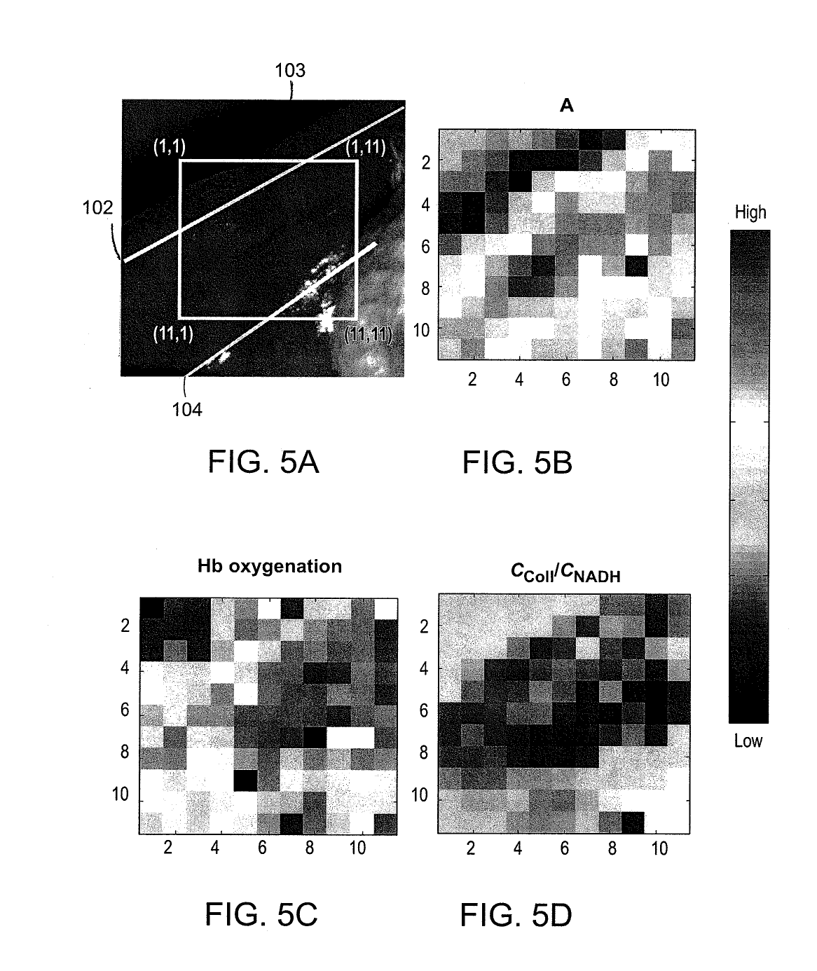

Wide area coverage is achieved by means of a 2D scanning mirror 64 (OIM102, Optics in Motion LLC), which can tilt by up to ±1.5° along two orthogonal axes, and thus raster scan the diagnostic spot across a 2.1 cm×2.1 cm region of the tissue surface in a stepwise fashion. At each mirror position, a reflectance measurement is made, followed immediately by a fluorescence measurement, I. Georgakoudi et al., “Fluorescence, reflectance, and light-scattering spectroscopy for evaluating dysplasia in patients with Barrett's esophagus,” Gastroenterology 120, 1620-1629 (2001) and I. Georgakoudi et al., “Trimodal spectroscopy for the detection and characterization of cervical precancers in vivo,” American Journal of Obstetrics and Gynecology 186, 374-382 (2002) and as further described in U.S. Pat. No. 6,912,412, the entire contents of these publications and the patent being incorporated herein by reference. Each pair of measurements is associated with a tissue location which correlates with the mirror position. However, this correlation can be affected by patient movement during the procedure. Therefore, the patient's movement needs to be tracked in order to take any shift in tissue position into account. An onboard color video camera 80 (QICAM, Qlmaging Corp.) tracks the patient movement relative to the instrument by acquiring one photograph of the tissue every second during the procedure using a zoom lens 82 and beamsplitter 74. Two white light LEDs (CCS Inc.), which provide extra illumination for the video camera, are turned off during reflectance and fluorescence measurements. A perspective view of the optical delivery and collection system in FIG. 6 where a white light delivery fiber 61 can be coupled separately from laser delivery fiber 63.

Measurements have been conducted with phantoms to determine the impact of different working distances on the DRS and IFS measurements. From 21.5 cm to 23.5 cm, it was found that the extracted parameters (A, B, etc.) varied by less than 10% of the values measured at 22.5 cm (the optimal working distance). Since the color video camera and the white light LEDs allow us to place the instrument very close to the optimal working distance (well within the +1 cm range studied with phantoms) prior to acquiring data, the instrument always operates very close to the optimal working distance. The total time required for a 2.1 cm×2.1 cm scan is approximately 90 s at present, but this time can be considerably reduced by using a CCD camera (for spectra) with the on-board memory (to eliminate the data transfer time and greatly reduce the data writing time), and increasing the angle of optical collection (to decrease the CCD exposure time).

After the procedure, the phase correlation technique is applied to determine the lateral translation relative to the instrument of each photograph relative to the first photograph. This technique involves computing the cross-correlation of two images and identifying the coordinates of the maximum value. The coordinates are the two coordinates of lateral translation. Phase correlation in the QSI instrument determines the two coordinates of translation within 0.2 mm of their true values. Applying phase correlation to each photograph, the system tracks the lateral shift in tissue position at the time of each photograph. This information is then used to correct the interrogated tissue location. The present system does not account for other types of motion such as rotation or tissue morphing because they are considerably less significant than translation, however, these can be corrected for using a sensor to detect tissue motions and provide feedback control of the motions of the scanning mirrors.

Physical tissue models were used (“phantoms”) with known scattering, absorption and fluorescence parameters to calibrate the QSI system and establish its accuracy. These phantoms consist of mixtures of 10% intralipid (Fresenius Kabi AG), hemoglobin (Sigma Aldrich Co.), water, and furan (Lambda Physik) at various concentrations. Furan is a fluorescent dye with excitation and emission spectra similar to that of collagen, an endogenous tissue fluorophore. Spectralon (Labsphere SRS-20) was used as a reflectance standard. Measurements were made over the spectral range 387-707 nm.

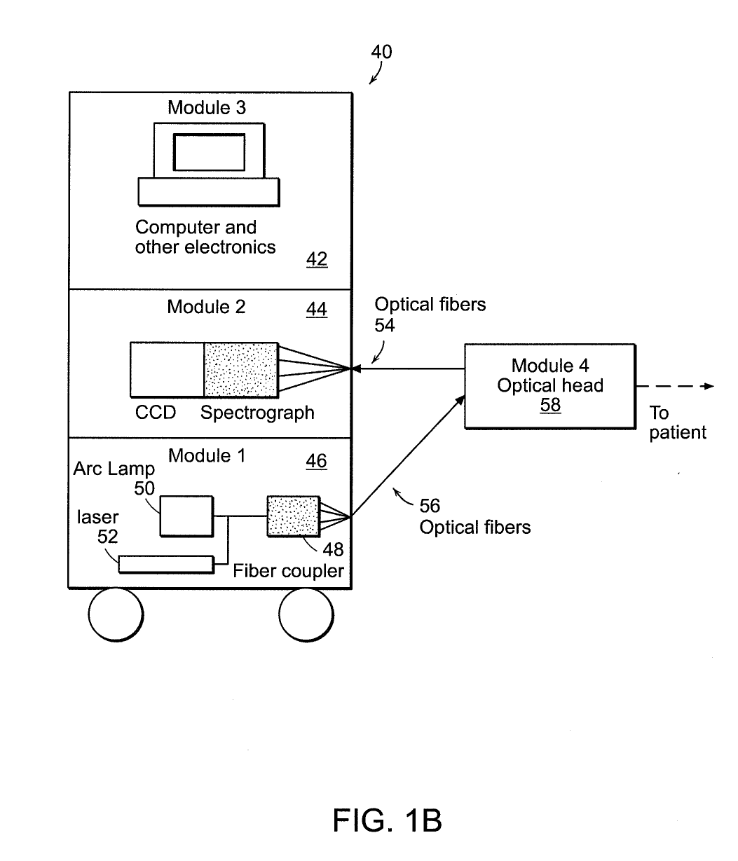

To measure the accuracy of the QSI system's reflectance measurement capability, nine combinations of intralipid diluted with water and hemoglobin. The three dilution ratios (with corresponding mass concentrations in parentheses) for intralipid were 1:9 (1%), 2:8 (2%), and 3:7 (3%). The three concentrations of hemoglobin were 0.5, 1.0, and 1.5 mg/mL. We define the interrogated spot as a “pixel.” The reflectance spectra were collected from phantoms, over 441 pixels over a 2.1 cm×2.1 cm region, were normalized by the corresponding reflectance spectra measured from the spectralon to remove spectral distortions and spatial inhomogeneity due to the instrument's spectral and spatial responses. The solid lines of FIG. 2 show the resulting calibrated reflectance spectra taken from the same imaging position on different phantoms 90, 92 and 94. DRS is used to extract diagnostic information: Each reflectance spectrum is fit (dashed lines) to the model described by Zonios et al., referenced above, to yield the reduced scattering μₛ′(λ) and absorption μₐ(λ) coefficients of the pixels, with μₛ′ (λ) and μₐ (λ) of the form:

$${μ}_{s}^{′} ( λ ) = {A ( \frac{λ}{{λ}_{0}} )}^{- B} , \\ {μ}_{a} ( λ ) = 0.23 {c}_{Hb} ( ( 1 - α ) {ɛ}_{Hb} ( λ ) + α {ɛ}_{{HbO}_{2}} ( λ ) ) . \qquad ( 1 )$$

For each pixel, four DRS parameters are extracted: A, the reduced scattering coefficient at the reference wavelength (i.e. A=μₛ′(λ₀), with λ₀=700 nm); the exponent, B, related to the average scatter size; and c_{Hb} and α, the concentration (mg/mL) and oxygen saturation of hemoglobin, respectively. ε_{Hb}(λ) and ε_{HbO}_{₂}(λ) are the extinction coefficients of 1 mg/mL of deoxygenated and oxygenated hemoglobin, respectively. As can be seen, the agreement between the measured and fitted spectra is excellent.

After extracting the four parameters, parameter maps across the 2.1 cm×2.1 cm region were obtained with a spatial resolution of 1 mm×1 mm. Table 1 summaries the extracted values for parameters A, B, and c_{Hb}. The mean value of A changes linearly with Intralipid concentration, as expected. The mean for c_{Hb} tracks the expected value with less than 10% difference. The mean values for A and B vary little with hemoglobin concentration, and the mean values for c_{Hb} vary little with intralipid concentration, indicating that QSI successfully decouples scattering from absorption. The standard deviations of the parameters across each phantom are less than 5% of the mean value, indicating a 5% variation in measured parameter values by QSI.

The above phantom measurements establish that A measurements scale accurately, and do not address the absolute accuracy of A and B measurements. This is because the scattering properties of the batch of intralipid used were not known precisely. The optical properties of intralipid have been characterized, but from experience, these properties vary considerably from batch to batch. To obtain the absolute accuracies of A and B measurements, QSI is used to measure a phantom consisting of 1 μm diameter polystyrene spheres (Polysciences, Inc.) in water with number density 1.1×10¹⁰ spheres/mL. For this phantom, the reduced scattering coefficient can be computed with Mie theory. To evaluate the absolute accuracy of QSI's A and B measurements, we fit the μₛ′(λ) computed with Mie theory to the reduced scattering coefficient from

$${μ}_{s}^{′} ( λ ) = {{A}_{Mie} ( \frac{λ}{{λ}_{0}} )}^{- {B}_{Mie}}$$

and obtain A_{Mie}=2.23 mm¹ and B_{Mie}=0.93. We then use QSI to conduct a reflectance measurement on the phantom, using the same procedures as with the nine intralipid phantoms above, and obtain mean values of A and B equal to 2.12 mm⁻¹ and 1.06, respectively. The excellent agreement of the parameters measured by DRS with the input parameters in both types of phantoms indicates that our system is properly calibrated.

TABLE 1
Reflectance parameters measured from tissue phantoms. The
uncertainty of each parameter is the standard deviation of
values measured from all 441 pixels on the phantom. Phantoms
labeled (a), (b), and (c) are included in FIG. 2.

| | Hemoglobin | | | |
|---|---|---|---|---|
| | concen- | | | |
| Intralipid mass | tration | | A (λ₀ = | |
| concentration | (mg/mL) | c_{Hb} (mg/mL) | 700 nm) | B (λ in nm) |
| 1% (c) | 0.5 | 0.53 ± 0.01 | 1.13 ± 0.01 | 0.99 ± 0.02 |
| 1% | 1.0 | 0.96 ± 0.01 | 1.13 ± 0.01 | 1.00 ± 0.02 |
| 1% | 1.5 | 1.56 ± 0.03 | 1.11 ± 0.01 | 1.02 ± 0.02 |
| 2% | 0.5 | 0.55 ± 0.01 | 1.90 ± 0.02 | 1.32 ± 0.04 |
| 2% (b) | 1.0 | 1.04 ± 0.01 | 1.86 ± 0.02 | 1.36 ± 0.04 |
| 2% | 1.5 | 1.49 ± 0.01 | 1.86 ± 0.02 | 1.40 ± 0.04 |
| 3% | 0.5 | 0.53 ± 0.01 | 2.67 ± 0.04 | 1.38 ± 0.04 |
| 3% | 1.0 | 1.05 ± 0.01 | 2.63 ± 0.04 | 1.45 ± 0.04 |
| 3% (a) | 1.5 | 1.50 ± 0.01 | 2.61 ± 0.03 | 1.48 ± 0.04 |

To determine the accuracy of fluorescence measurements, we prepared six combinations of intralipid diluted with water, hemoglobin, and furan were prepared. IFS is used to analyze the fluorescence for each pixel: Reflectance and bulk fluorescence spectra are measured from 441 spots on each phantom. The extracted reduced scattering and absorption coefficients are used to correct the bulk fluorescence spectra using the model described by Müller et al., referenced above, to extract the IFS spectra, which are the signals that would be measured in the absence of scattering and absorption.

TABLE 2
Fluorescence parameters measured from tissue phantoms.
The measured furan concentration for 1% intralipid has been
normalized to 0.25 and all other measured concentrations are
normalized by the same factor.

| | Hemoglobin | Prepared furan | |
|---|---|---|---|
| Intralipid Mass | concentration | concentration | Measured furan |
| concentration | (mg/mL) | (μg/mL) | concentration |
| 1% (a) | 0.5 | 0.25 | 0.25 ± 0.004 |
| 2% (b) | 0.5 | 0.25 | 0.26 ± 0.005 |
| 2% | 1.0 | 0.25 | 0.23 ± 0.004 |
| 2% | 0.5 | 0.125 | 0.11 ± 0.004 |
| 2% | 0.5 | 0.5 | 0.47 ± 0.006 |
| 3% (c) | 0.5 | 0.25 | 0.26 ± 0.005 |

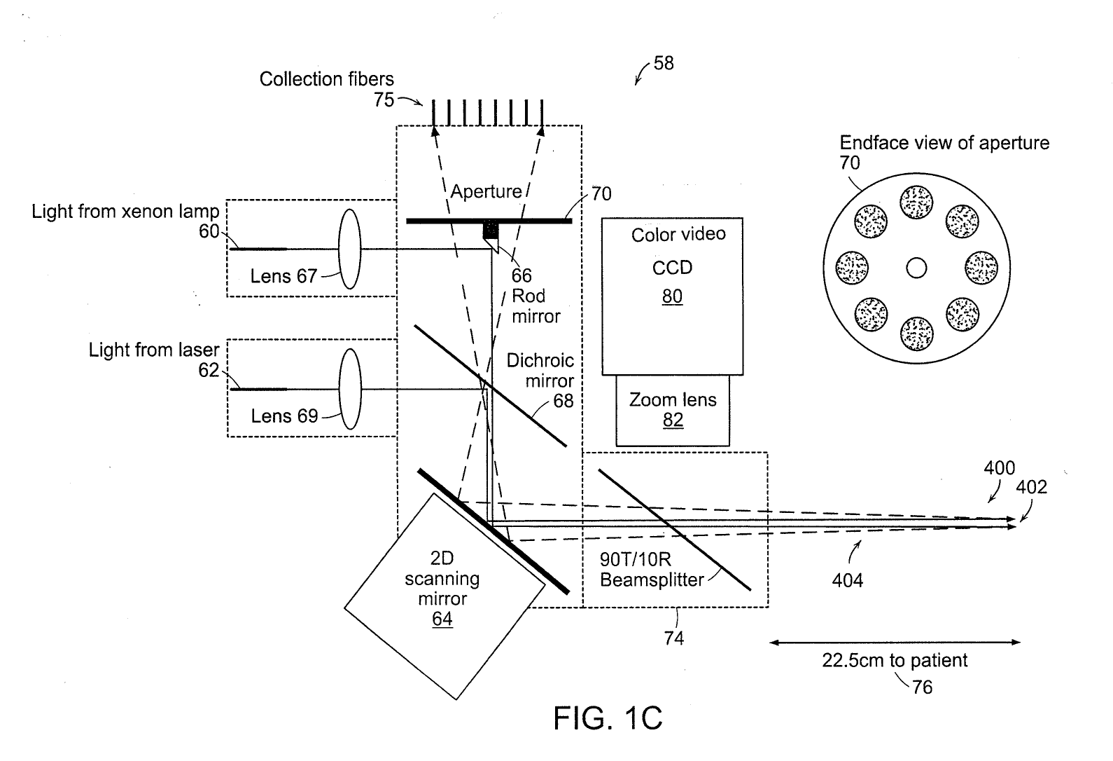

A fluorescence spectrum of furan in water excited by 337 nm light as the basic spectrum for our fluorescence calibration. The instrument collection spectral response, which is extracted by taking the ratio between the basic spectrum and the measured spectrum of furan in water, is taken into account in all our fluorescence spectra shown in this paper. FIG. 3 shows several bulk and IFS 95 spectra measured from on different phantoms (with hemoglobin and furan concentrations fixed but the intralipid concentration varied from 1% to 3%). The data indicates that bulk fluorescence spectra vary considerably with intralipid concentration while the IFS spectra do not, as expected. The intrinsic fluorescence spectra of FIG. 3 are the same as the fluorescence spectrum of furan in water excited by 337 nm light, which is shown in line 98 in FIG. 3. This agreement indicates that IFS can be used to remove the distortion caused by tissue scattering and absorption and extract intrinsic fluorescence spectra.

Using one of the IFS spectra and the known concentration as a standard, the furan concentrations were measured in each pixel of each phantom by recording the amplitude difference between the spectrum from the pixel and the basis spectrum.

Table 2 shows the average furan concentrations measured from the 441 pixels on each phantom. The agreement between the prepared and measured furan concentrations is excellent. The variation in parameters measured across the homogeneous phantom is less than 5%. Therefore, 5% is a measure of the smallest difference between fluorescence properties measured from neighboring points of an inhomogeneous sample that can be resolved by the present QSI instrument.

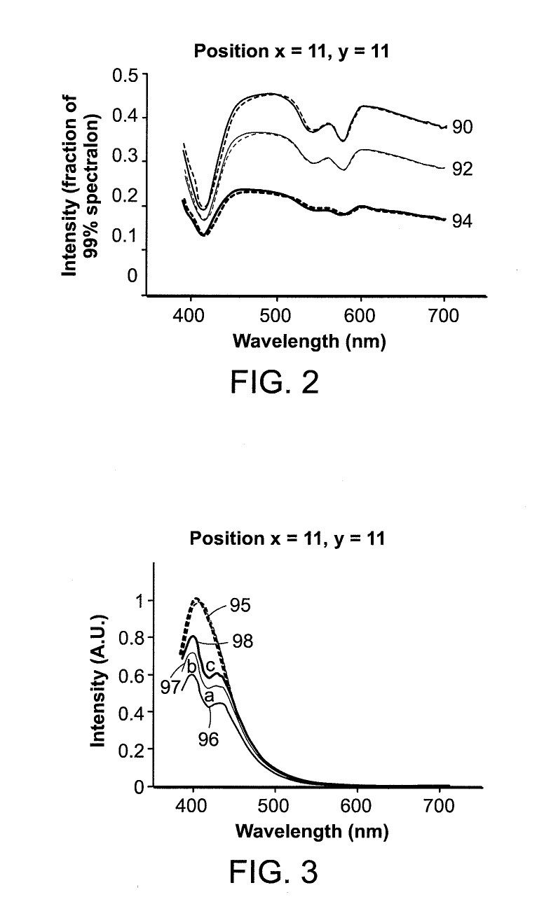

The ability to detect cancer using the QSI system was demonstrated using an ex vivo colon cancer specimen. Four histological sections were made at ˜4 mm intervals through the tissue (indicated by dashed lines in FIG. 4A, all of which showed similar pathological features, FIG. 4B is one representative section. Given that the lesion was macroscopically contiguous, it was assumed that these 4 sections represent the entire lesion. Boundaries of the entire lesion were thus defined by interpolation of histopathological features between the 4 actual histological sections. The area below the line 100 was classified as the region of colon cancer. The area investigated by QSI (enclosed by the white square 101) was 2 cm by 2 cm with 400 pixels. From each pixel, we extracted four DRS parameters (A, B, c_{Hb} and α) and two fluorescence parameters (collagen concentration, c_{Coll} and NADH concentration, c_{NADH}). To measure the concentrations of two fluorophores at each point on the tissue requires a modification of the approach used for the tissue phantoms. As before, the IFS spectra were first extracted from the data. These spectra are a linear combination of collagen and NADH fluorescence. To extract the relative concentrations, the fluorescence basis spectra of collagen and NADH were determined by applying multivariate curve resolution, with the 400 intrinsic fluorescence spectra measured from the sample. Fluorescence from the two fluorophores can then be linearly combined to yield the net fluorescence. To obtain the best fit, the following equation is optimized over all wavelengths and at all points.

$${I}_{data} ( x , y , λ ) = {\sum }_{i = 1}^{2} {c}_{i} ( x , y ) {I}_{basis}^{i} ( λ ) . \qquad ( 2 )$$

Here, I_{data}(x,y) is the measured intrinsic fluorescence spectrum from point (x, y); I_{basis} are the basis spectra of collagen and NADH. I_{basis} is normalized such that its peak emission is 1. c(x,y) are the concentrations of collagen and NADH at point (x, y).

Bayes' theorem was used to develop a classification algorithm to distinguish between normal and cancer groups. The training set consisted of 16 normal and 16 cancer data points chosen from two equal sized regions of the sample located away from the boundary between normal and cancer (FIG. 4 FIG. 4A). A two-tailed Student's t-test was used to compare extracted spectroscopy parameters between normal and cancer groups. A p-value <0.05 was considered significant. It was found that the three most significant parameters to differentiate between the normal and cancer groups are A, c_{Hb}, and the c_{Coll}/c_{NADH} ratio. The maps for these parameters are shown in FIGS. 4C, 4D and 4E. These parameters represent three different types of tissue information: structure, hemoglobin absorption, and biochemistry, respectively. Multivariate Gaussian probability distributions were used to model the data. Bayes' rule was applied prospectively to calculate the posterior probability of cancer (“pc”) for each of the imaged pixels. The prior probablity was estimated from the pathology image to be 0.5. FIG. 4F represents the resulting diagnostic map, constructed by using the threshold for assigning a pixel to the cancer group of pc>0.5. The QSI classification is consistent with the histological classification of FIG. 4A, and it demonstrates the ability of our QSI system to distinguish between cancer and normal tissue in this specimen.

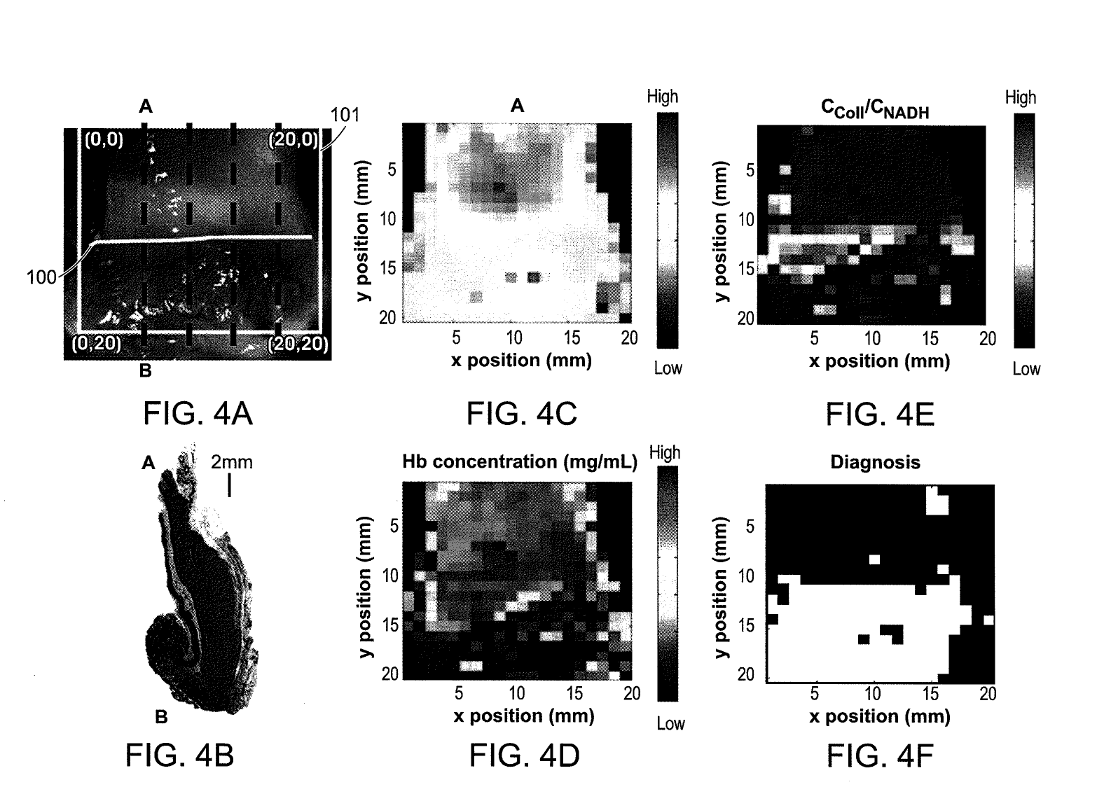

The QSI system was then used to examine a suspicious tissue site, as determined by the physician using conventional white light examination, on the ventral tongue of a patient. This was the first in vivo measurement conducted with the QSI system, and more will be conducted in the near future. The purpose of this study is to demonstrate the in vivo applicability of the QSI instrument instead of making correlations. FIG. 5A is the white light image of the ventral tongue. The lines 102, 104 indicate the boundaries between normal areas and a region of hyperkeratosis identified by the oral surgeon. Subsequent histopathology analysis of biopsied tissue determined the site to be a benign area of hyperplasia. The gauze (shown on the lower right corner) was used by the oral surgeon to hold the tongue in order to reveal the hyperkeratosis for scanning. The tissue within the white box 103 was interrogated with QSI system.

FIGS. 5B-5D show reflectance and fluorescence parameters measured from 121 points on the site over a 1.1 cm×1.1 cm area in approximately 15 s. FIG. 5B indicates an increase in scattering in the hyperkeratotic region as compared to the surrounding normal mucosa. FIG. 5B shows reductions in c_{Coll}/C_{NADH} in the hyperkeratotic region. This is consistent with the understanding that keratin reduces the amount of light reaching the stroma, thus reducing collagen fluorescence. This study represents our first in vivo measurement. Detailed comparisons between spectroscopy and histology in multiple patients in various tissue types can be used to provide accurate diagnostic image information, as well as insights into the correlation between QSI parameters and tissue state.

Current cancer diagnosis often employs visual inspection of a wide area of tissue (sometimes assisted by endoscopy), followed by biopsy of suspicious sites. As mentioned in the introduction, this leads to unnecessary biopsies and delays and inaccuracies in pathology. Quantitative spectroscopy (QS), which combines DRS and IFS, for early cancer detection, seeks to address those shortcomings by extracting parameters that characterize a tissue sample without tissue removal. The extracted parameters can then be combined to form a diagnostic algorithm to quantify the probability of the interrogated tissue being dysplastic. The analysis can be performed by computer in real time. Although QS as a contact probe technique is effective in various organs, it is essential to extend it to the imaging mode so that wide areas of tissues can be studied.

The present invention provides an extension of QS to the imaging mode. QS methodology is a model-based approach that extracts tissue morphological and biochemical information from tissue reflectance (DRS) and fluorescence (IFS) spectra. The method is based on the correlation of tissue parameters with disease state. Spectral changes as used to develop algorithms for diagnosing cancer without analyzing the underlying tissue parameters, which can be used to understand tissue composition and chemical makeup. In contrast, QSI provides a method of analysis based on understanding the biochemical and morphological structure of the tissue. This information can provide more accurate and robust diagnosis.

Our spectral imaging approach of raster scanning small spots is distinct of those of other methods, most of which use full-field illumination. Although full-field illumination can be used for wide area detection using a CCD camera, it cannot be used to provide the desired quantitative information. Cross talk between spatial locations can occur, so the information extracted from one location can be influenced by neighboring locations. UV excitation power densities are small since the light is distributed over the entire area. This imaging significantly limits the speed at which fluorescence measurements can be performed. Moreover, there is a fundamental reason why raster scanning is employed rather than full-field illumination. To extract tissue parameters, A, B, c_{Hb} and a in Eq. (1), the measurement of the reduced scattering λₛ′(λ) and absorption μₐ(λ) coefficients are measured independently. However, with full field illumination, only the ratio of μₛ′(λ) and μₐ(λ) can be measured. Use of a probe with small delivery and collection spot size provides a scale parameter r_{c}′, for the measurement, which provides information about two dimensionless parameters μs′(λ)*r_{c}′ and μₐ(λ)*r_{c}′ measured at each pixel.

This system accomplishes wide field coverage by employing a virtual probe to sample a small area (defined as one pixel) of tissue at any one time. As discussed above, this probe has effectively all of the features of a contact probe, without the need to make contact with the tissue. Raster scanning is used to cover a large area of tissue, pixel by pixel. This would not be possible with a contact probe. Because the contact-probe feature of fixed delivery-collection geometry is used, this method allows us to directly transfer the data analysis procedures and results obtained from our contact-probe studies to our imaging studies. This is the first spectral imaging system capable of extracting tissue biochemical and morphological information quantitatively.

The system has been used to demonstrate accurate extraction of tissue parameters using physical tissue models (“phantoms”). The ability of our QSI imaging system to identify cancerous lesions was demonstrated on excised tissue, and in vivo use of this imaging system was demonstrated in the oral cavity.

Multispectral autofluorescence and reflectance images of the cervix are acquired using an inexpensive color CCD camera for in vivo detection of cervical cancer. Spectral sensitivity is provided by the three color channels of the CCD. Also an acousto-optic tunable filter (AOTF) can be used to select the wavelength of the tissue fluorescence image acquired by a CCD. This AOTF-based spectral imaging system can record spectral images at a series of wavelengths of interest to provide spectral contracts for diagnosis. A spectral imaging system using a liquid-crystal tunable filter (LCTF) to select wavelength of the tissue images acquired by a CCD. Both reflectance and fluorescence spectral contrasts were shown between cortex and white matter on the in vitro mouse brain and between tumor and normal cortex on the in vivo human brain. All three systems mentioned above used full-field illumination and collection with different types of wavelength selection mechanisms. The spectral imaging system can be used for cervical tissue, and uses different illumination/collection geometries for measuring diffuse reflectance and fluorescence spectra consecutively from a 1 mm interrogation region. For reflectance, full-field illumination was used, whereas fluorescence was implemented by delivering a 1 mm diameter spot of UV excitation and collecting fluorescence from the same location. Full cervical scans employed 499 interrogation locations. Spectral differences were observed between normal squamous epithelium and high-grade cervical intraepithelial neoplasia.

All of these spectral imaging systems reported observation of spectral contrast between benign and malignant tissues in reflectance and/or fluorescence. Some of these systems also demonstrated the effectiveness of using this contrast in tissue diagnosis.

However, there are further advances using quantitative spectral imaging modalities such as QSI for detecting early cancer noninvasively and objectively. QSI extracts quantitative information about morphological and biochemical tissue constituents that give rise to spectral contrast. This provides several important benefits: (1) Because the extracted parameters are tissue properties, the results are more robust and instrument independent; (2) the extracted parameters have physical meaning which can be correlated with morphological and biochemical information. Since the information extracted is intrinsic to the tissue, the diagnostic algorithm developed using tissue parameters are more robust and can be directly transferred from contact probe application to imaging mode. This is true even though the illumination and collection geometries may differ, as such difference can be taken into account in the modeling. Few changes are needed for tissue parameter extraction except for instrument-dependent constants such as effective probe radius r_{c}′ and the wavelength range used.

QSI uses tissue parameters for contrast to give diagnoses. Knowledge about morphology and biochemistry of cancer as it evolves can be used to develop QSI algorithms and authenticate their validity. As an example, collagen in the tissue matrix degrades in cancerous tissue. Therefore, the scattering intensity for cancerous tissue should decrease, which is consistent with our DRS images (FIG. 4C). It is also known that higher NADH concentration accompanies cancer progression (since NADH is a measure of intracellular oxidation-reduction states in vivo), due to greater metabolic activity of cancerous cells. Therefore, the concentration ratio between collagen and NADH should be lower for cancerous tissue, which is again confirmed by the IFS images in the ex vivo study (FIG. 4F). Moreover, combining DRS and IFS provides more complete tissue morphological and biochemical information, which can be advantageous in cancer detection.

This system has demonstrated the extension of quantitative spectroscopy from a contact probe modality to a wide-area imaging technique. Its ability to provide spectral contrast based on tissue parameters has been demonstrated in the example of ex vivo colonic tissue. In addition, QSI's ability to measure spectra in vivo has been established. The current QSI instrument is designed to image openly accessible sites such as the cervix, oral cavity and skin. QSI can also be applied in an endoscope-type delivery system to image the hollow organs of the body. This offers the potential of using the quantitative diagnostic ability of spectroscopy to diagnose cancer, atherosclerosis, and other disease states throughout the body. In addition, QSI can readily be used to conduct margin detection. This will potentially reduce the amount of time the patient and medical staff have to wait for results from pathology and the number of return visits required.

The present invention can be used for clinical in vivo measurements for imaging of cervical dysplasia using the QSI instrument as well as with an endoscopic imaging system for other internal body tissues.

An endoscopic QSI system which has several important advantages including a miniaturized QSI optical head diameter; a faster data acquisition time of 0.3 second for DRS measurement from an area 6 mm in diameter; and near-continuous monitoring capability. All three features are essential for implementing QSI in endoscopic configurations.

The main functions of the optical head shown in FIG. 6 are light delivery 400 (white light for DRS and UV light for IFS) to the diagnostic spot 402, collection 404 from the diagnostic spot, and monitoring by the camera 80 for positioning the instrument and motion compensation. The scanning of the diagnostic spot on the tissue is achieved by the scanning mirror 64 which is built into the optical head. The size of this optical head is about 9 inches (L) by 6 inches (W) by 18 inches (H). The data acquisition time from an area 2.1 cm by 2.1 cm is about 90 seconds. There is no near-continuous monitoring feature during the DRS or IFS measurements.

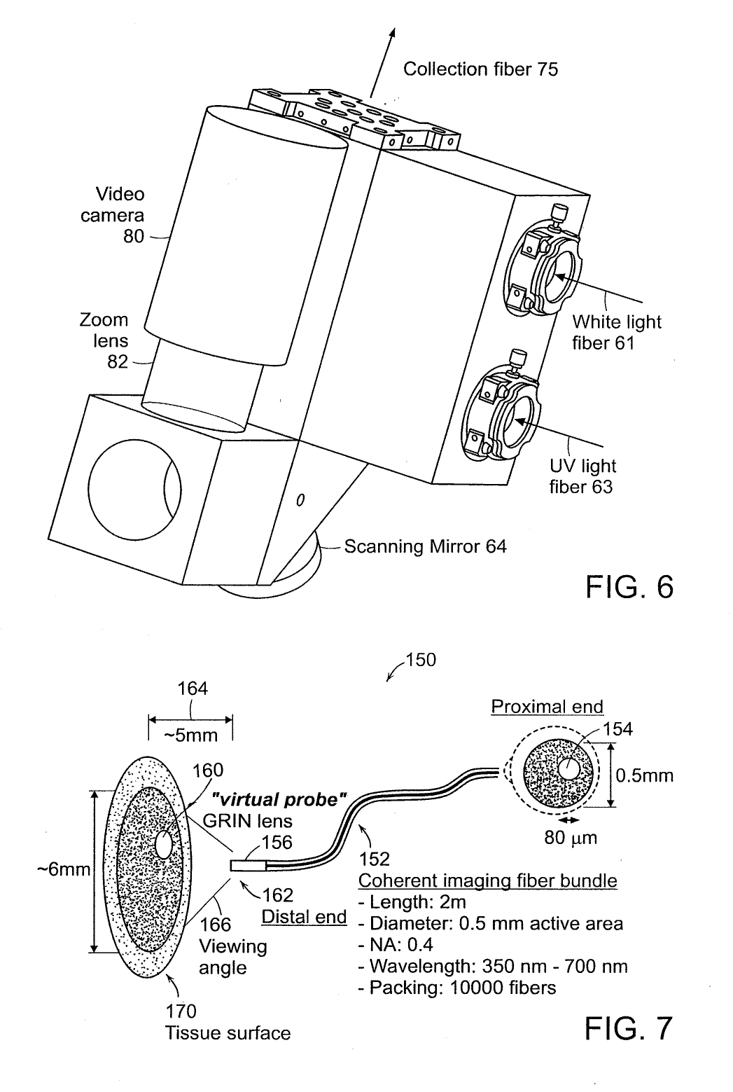

FIG. 7 depicts the endoscopic QSI system 150 spot scanning, note that the specific system presented here represents those of one fiberscope used and these numbers can be changed to adapt to different application requirements. The light delivery and collection region 154 (with a diameter of 80 μm, on the proximal end) is relayed via the coherent imaging fiber bundle 152 (Fujikura, 0.5 mm diameter, 10,000 pixels) to the distal end 162 with a gradient index (GRIN) lens 156 (also 0.5 mm diameter) attached. The GRIN lens serves as the objective lens, which projects (with a magnification of 12.5) the virtual probe image (diameter 80 μm, on the distal end) onto the tissue surface to be the virtual probe with a diameter of ˜1 mm. The tissue 170 and the fiberscope are separated by a working distance 164 of 5 mm, in this embodiment. The viewing angle 166 is 63 degrees. The exact location of the virtual probe image on the proximal end of the fiber is determined by a 2D scanning mirror, which, in turn, allows scanning of the light region 160 across the tissue surface.

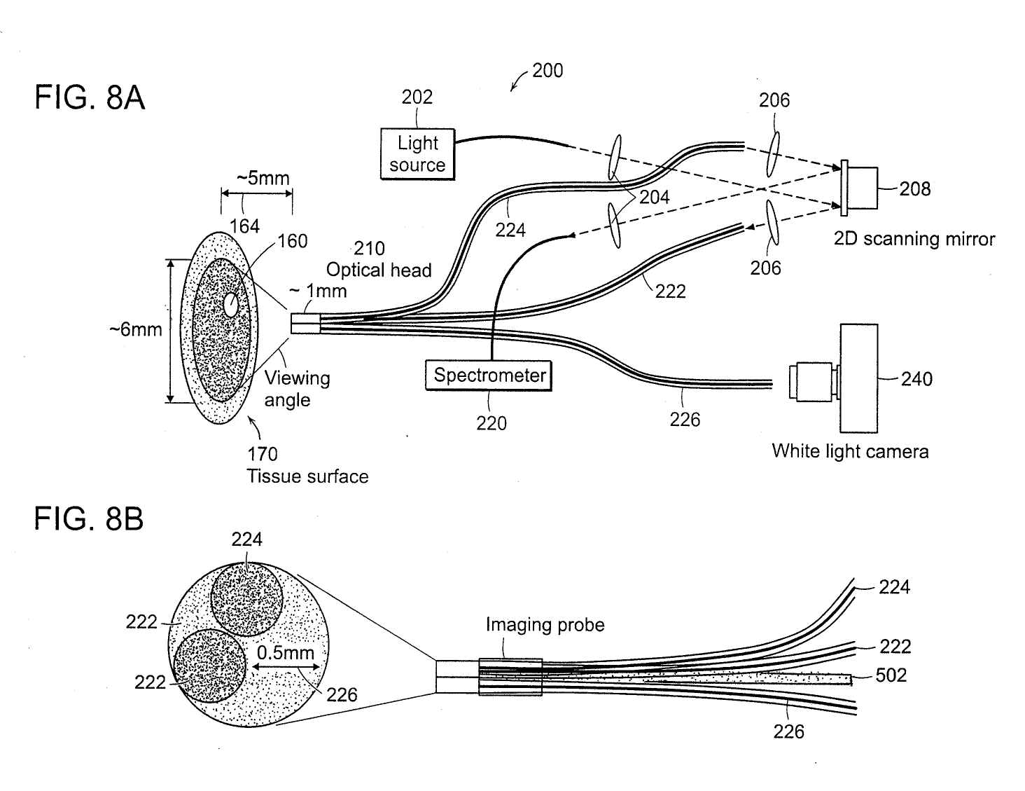

FIG. 8A is the schematic diagram of the endoscopic QSI system. DRS is implemented to carry out the miniaturization and improve the speed of the DRS data acquisition for the endoscopic QSI system and demonstrate the continuous monitoring feature.

In the embodiment of FIG. 7, only one fiberscope is needed for both the delivery and collection. In FIGS. 8A and 8B, two separate fiberscopes for the delivery 222 and collection for preventing the back reflection of the delivery fiber (from the GRIN lens-air interface) from going into the collection fiber; and for increasing the flexibility in the sizes of the delivery and collection and their separation. In addition, one fiberscope as a monitoring channel is also added. Thus, the optical head consists of three fiberscopes: delivery 222, collection 224, and monitoring 226. The delivery and collection fiberscopes can be identical, whereas the monitoring fiberscope has a larger viewing angle of 74 degrees as the only difference. The same field of view (because of the same viewing angle) with a diameter of 6.1 mm for delivery and collection enables matching the delivery and collection light paths. The larger field of view (with a diameter of 7.5 mm) for the monitoring with camera 240 is to guarantee the monitoring field of view covers the entire QSI field of view. Note that the diameter of the optical head is about 1 mm which is well suited for endoscopic configurations, in which the distal end can be inserted through the working channel of an endoscope. A single scanning mirror 208 or several can be used.

White light from the light source (CW 300W Simplicity series, Newport Corp.) is coupled into the delivery single-core fiber (0.22 NA, 200 μm core, Thorlabs M25L01). The 200 μm light spot on the other end of the fiber is imaged to a 80 μm diameter light spot on the proximal end of the delivery fiberscope by using two UV/VIS objective lenses (Olympus UPLSAPO 10× with 0.4 NA and UPLSAPO 4× with 0.16 NA). The delivery fiberscope relays this delivery spot unto the tissue surface with a magnification of 12.5. Consequently, the delivery for the virtual probe (shown as a white spot on the tissue surface in FIG. 8A has a diameter of ˜1 mm. The tissue reflectance (or fluorescence) from the same spot is relayed by the collection fiberscope with a demagnification of 12.5 to a light spot (with a diameter of 80 μm) on the proximal end (of the collection fiberscope) which is then coupled into the collection single fiber (0.22 NA, 200 μm core, Thorlabs M25L01) using two UV/VIS objective lenses (Olympus UPLSAPO 10× with 0.4 NA and UPLSAPO 4× with 0.16 NA). Finally, the tissue reflectance is coupled into the spectrometer which includes a spectrograph (SP150, Princeton Instruments) and monochrome CCD (PhotonMAX, Princeton Instruments). Coupling optics such as lens 504 can be used for optical delivery to scanner 208. The diffuse reflectance spectrum is then recorded. The pair of spots, delivery and collection, on the tissue surface form the virtual probe where the tissue is interrogated by quantitative spectroscopy. By changing the angle of the 2D scanning mirror (OIM102, Optics in Motion LLC), both the delivery and collection are scanned concurrently on the proximal ends of the delivery and collection fiberscopes, respectively. As a result, the virtual probe is scanned over the tissue surface to cover a wide area (with diameter up to ˜6 mm) to perform QSI measurements. LED source 520 can be coupled using fiber 502. The distal ends are show in FIG. 8B.

While the probe is scanning the diagnosed tissue area, the monitoring CCD (Lumenera, INFINITY 2-1) for the endoscopic QSI system records a video with 50 ms exposure per frame and 20 frames per second. Therefore, the recorded video has the full record of the data acquisition and can locate the positions of where all the spectra are taken. This monitoring feature is a significant improvement over the current monitoring method used by the free-space QSI system. Note that during the data acquisition, only the illumination for the probe is on. Therefore, this monitoring is performed without affecting the DRS measurements.

The optical head needs to perform three functions (delivery, collection, and monitoring) and also needs to be small (−1 mm) in order to implement QSI in the endoscopic configuration. FIG. 8 shows the optical head can include three fiberscopes. The size of the optical head is in a range of 1-4 mm in diameter.

FIGS. 8A and 8B demonstrates the continuous monitoring capability of the endoscopic QSI system. The monitoring field of view is about 7.5 mm diameter. The endoscopic QSI system has two operation modes: white-light endoscope mode and DRS mode. In the white-light endoscope mode, it works the same as a medical endoscope. The white-light used in the endoscope mode was turned off when the DRS measurement was performed. In the DRS mode, the monitoring CCD was recording a video with the 50 ms exposure time for each frame and 20 frames per second. Consecutive frames cover the whole span of the DRS measurement. The probe was scanning counter-clockwise in 100 steps with 3 ms per step. The scanned circular area was about 6 mm in diameter.

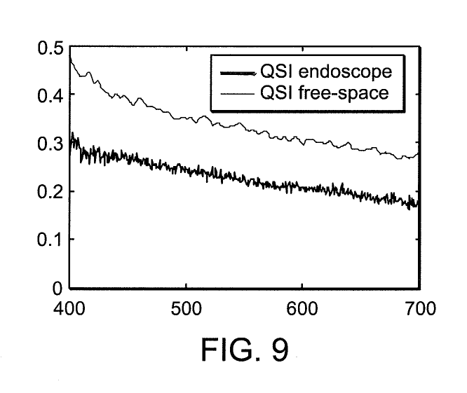

To compare the collection efficiency of the endoscopic QSI system to that of the free-space QSI system, we conducted two reflectance measurements using those two QSI systems. The illumination power was similar for both systems. Therefore, the signal difference was from collection which is a function of collection NA and exposure time. The collection solid angle ratio due to NA difference is 12.5 (endoscope):1 (free-space). The exposure times used were 3 ms (endoscope) and 50 ms (free-space). Therefore, the predicted signal ratio is estimated to be ˜3 (endoscope):4 (free-space). Two spectra shown in FIG. 9 are the reflectance spectra measured from the tissue phantom consisting of 1 μm diameter polystyrene spheres (Polysciences, Inc.) in water with number density 1.1×10¹⁰ spheres/cc using the free-space QSI system (upper curve) and the endoscopic QSI system (lower curve). This confirmed the endoscopic QSI system is capable of measuring DRS with a comparable signal size but much less exposure time because of its higher collection efficiency. The exposure time used for the endoscopic QSI system was 3 ms which matched the time per step for spiral scanning. Therefore, the whole DRS measurement, covering a circular area of 6 mm in diameter, spans 0.3 seconds. This demonstrates the endoscopic QSI system can measure DRS with fast data acquisition which is important for the endoscopic implementation of QSI.

The system can employ image processing to utilize tissue spatial contiguity, which can provide additional diagnostic information not available in the contact probe configuration.

Spectral images can be collected from cancerous colon tissue ex vivo and suspicious oral tissue in vivo. The methods were applied to obtain quantitative information about tissue physiology. In the oral cavity, changes in stromal density and hemoglobin concentration have been measured with spectroscopy and correlated to cancer progression. However, the method relies on diffusion theory being an accurate description of light propagation, which may not be the case in all tissue types as we saw with the tissue phantoms and ex vivo colon. A model for DRS in samples where diffusion theory breaks down for extracting more accurate tissue scattering and absorption properties which are required for the correct extraction of the IFS spectra.

The Zonios' method for modeling elastic scattering and absorption by tissue is the key to the entire quantitative spectroscopy process because Müller's intrinsic fluorescence method for extracting fluorophore concentrations depends on accurate determination of the scattering and absorption coefficients. The Zonios' method simplifies the light diffusion model developed by Farrell et al. Previously, an inversion algorithm based on this method to extract the reduced scattering coefficient, μₛ′ and the absorption coefficient, μₐ. However, the Zonios' simplification, and the diffusion model as a whole, is accurate only when the following two conditions are satisfied: 1)/μₛ′>>μₐ and 2) the observation point is sufficiently far from sources and boundaries. The first condition is generally satisfied in the therapeutic window (μ˜650-950 nm) of most tissues, but can be strongly violated in the visible region of the spectrum. Further, the second condition may not be fulfilled in the endoscopic configuration where a small source-detection separation is necessary. In the instances where the diffusion model fails, a diffusion model-based inversion algorithm will not yield accurate values for μ₃′ or μₐ. Therefore, it is desirable to use an inversion algorithms that overcome these limitations. Two methods, Pₙ approximation and Monte Carlo-based inverse model, can be used. Both methods have merits. Pₙ provides physical insight and understanding into light propagation phenomena and is less computationally intensive. A Monte Carlo simulation can also provide accurate results.

The Boltzmann transport equation accurately describes light propagation in a scattering and absorbing medium such as tissue. However, analytical solutions to the equation do not exist for most instrument geometries. Without an analytical solution, solving the inverse problem to obtain scattering and absorption information from measured spectra becomes computationally very difficult. The P_{N} approximation is one technique for obtaining approximate analytical solutions to the transport equation. It involves expanding the angular dependent source and radiance terms in spherical harmonics and the scattering phase function in Legendre polynomials. P_{N} refers to the approximation made by truncating the expansions after N terms. Considering only steady state light propagation, P₁ is the diffusion approximation. P₁ is valid for large transport albedo a values and large source-detection separation. A typical criterion for a is that it must be greater than 0.98. Hull et al. have solved the P₃ approximation and determined that P₃ approximation models the radiance in highly absorbing media or close to sources more accurately than does diffusion theory and determined that it is accurate for source-detector separation greater than 0.43 mm and α>0.59. A preferred embodiment implements the P₃ approximation for the geometries of the current imaging instrument and the endoscopic imaging instrument. This can replace the Zonios' model for extracting scattering and absorption parameters from measured spectra. If extracted scattering and absorption coefficients violate the limits determined by Hull et al., higher order approximations can be used.

Photon transport in biological tissue can be numerically simulated by the Monte Carlo method. Considering light propagation in tissue one photon at a time, light energy collected by the instrument can be accurately predicted by Monte Carlo simulations. In Monte Carlo simulations, the propagation of a photon in the medium is traced from entry until exit. The distance a photon travels before encountering a scattering or absorption event is a random variable with cumulative distribution function

$$d = - \frac{ln ( 1 - x )}{( {u}_{s} + {u}_{a} )} ,$$

where d is the distance, x is a uniformly distributed random variable between 0 and 1, and μₛ is the scattering coefficient. The direction a photon travels in after a scattering event is a random variable with probability density function equal to its scattering phase function. If a photon exits within the collection area and solid angle of the instrument's collection optics, its energy can be measured. After simulating a large number of photons, the reflectance for any delivery-collection geometry can be predicted. Monte Carlo simulations are used to generate tables of reflectance for different scatterer sizes, densities, and indices of refraction and different absorber concentrations. These tables can replace the Zonios' model for extracting scatterer and absorber parameters from measurements.

A set of tissue phantoms spanning physiologically relevant reduced scattering and absorption coefficient ranges (0.7 mm⁻¹<μₛ′<3.3 mm⁻¹, 0 mm⁻¹<μₐ<2.0 mm⁻¹) prepared from various mixtures of intralipid and hemoglobin can be used for calibration. The reduced scattering coefficient range corresponds to an intralipid dilution range of approximately 1:20<dilution<1:5, where the dilution is of standard 10% intralipid in water.

Quantitative spectroscopy can be collected data from a single tissue point. Preferred embodiments of the present invention provide spectral imaging using the scanning approach employed by the current endoscopic instrument that collects data from multiple tissue points that are spatially adjacent. Imaging allows us to analyze parameter maps as spatial patterns rather than distinct data points. Since tissue features, including abnormal growths such as cancer, usually occupy a continuous region of tissue, parameter maps are processed to emphasize patterns that span numerous spatially connected pixels and deemphasize isolated pixels parameter values significantly different from that of its neighbors.

Two-dimensional low pass filtering can be used to emphasize tissue features and suppress artifacts. This is equivalent to signal processing in mechanical and electrical systems where true signals typically arrive at low frequencies and noise arrives at high frequencies. A Gaussian point spread function of finite width is convolved with each parameter map. In optics, this is equivalent to “blurring” an image by removing high spatial frequency components, which are typically non-physical in tissue. Convolution favors spatially continuous (low spatial frequency features) tissue patterns. FIG. 11 demonstrates this process on the A parameter map is interpolated to 1024×1024 pixels and convolved with a two-dimensional Gaussian point spread function, 2 mm FWHM. Pixels corresponding to sites without tissue are not included in the convolution. The convolution is binned back to 20×20 pixels. The resulting map emphasizes the normal and cancerous tissue regions.

Quantitative spectral imaging (QSI) is an imaging system which implements one or more spectral modalities, such as DRS and IFS, in the imaging mode to extract quantitative tissue morphological and biochemical information. QSI is employed in a clinical instrument that can be used to interrogate openly accessible organs such as the skin and the cervix. It can also be used to examine the oral cavity, the QSI system is configured for colposcopy and is thus similar in size to a colposcope and requires approximately 90 seconds to scan a 2 cm×2 cm region of tissue. The data acquisition time is proportional to the area inspected. Large instrument size and long acquisition times are sufficient for imaging openly accessible organs that can be held relatively still, such as the skin and cervix. However, many cancers originate in less accessible organs such as the colon, esophagus, larynx, vocal cords, nasal cavities, and mouth. Since these organs are not accustomed to foreign objects, inserting even a small endoscope can lead to reflex responses. As a result, to image these organs, small, flexible instruments with short acquisition times are required.

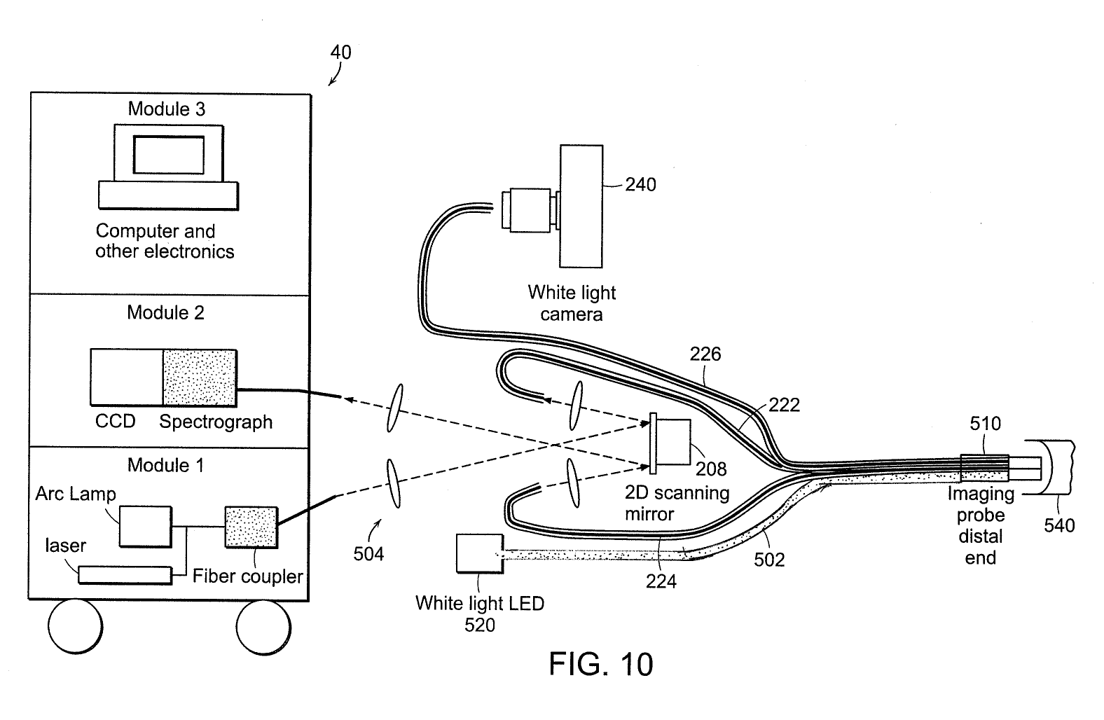

The component of the endoscopic QSI system is the imaging probe system shown in FIG. 10. By adding built-in illumination to the optical head, this illumination is used only for positioning the imaging probe before the DRS and IFS measurements. It can be turned off during the DRS and IFS measurements. FIG. 10 depicts the imaging probe distal end which can include four parts: delivery 222, collection 224, and monitoring 226 fiberscopes and an illumination fiber bundle 502. All four parts are tightly packed in a metal ferrule distal end 510 with a length of about 3 mm and an outside diameter of about 1.5 mm. The illumination fiber bundle 222, which is a bundle of multi component glass fibers (Fujikura, FMCF-77-50) with high NA (0.77) and small diameter (50 μm), fills the gaps between fiberscopes. With NA of 0.05, the depth of field of the monitoring fiberscope is ˜0.2 mm. Therefore, the user can position the imaging probe at 5±0.2 mm away from the tissue surface by optimizing the sharpness of the tissue image observed via the monitoring fiberscope. A therapeutic rhinolaryngofiberscope can be used for housing the imaging probe to access the larynx. Olympus ENF-T3 has a total length of 58.5 cm an instrument channel with a diameter of 2.2 mm. The endoscopic QSI system imaging probe, with a length of 2 meters and diameter of ˜1.5 mm, can be inserted through the instrument channel 540 to access the larynx.

FIG. 10 illustrates the design of the clinical endoscopic QSI system. The components (except UV laser light source) in Modules 1, 2, and 3, can be built in a cart, and can be identical to the ones used in the free-space QSI system described above. Because IFS spectra are acquired at a rate of approximately 1 kHz, the Nitrogen UV laser (NL100, Stanford Research Systems Inc., 20 Hz rep rate) used for the free-space QSI system is not appropriate. The user will replace it with a diode-pumped-solid-state laser (DPSS Lasers Inc., Model 3510-100, 355 nm, 1.0 W at 100 kHz rep rate). The optical head in the free-space QSI system (a) is separated into two parts in the endoscopic QSI system: Module 4 and imaging probe. Module 4, which can be built either in the cart or in a separate unit closer to the patient, consists of a 2D scanning mirror (OIM102, Optics in Motion LLC), a white light camera (Lumenera, INFINITY 2-1), and a white light emitting diode (LED, CCS Inc.). The white light LED is used for illuminating the tissue when positioning the image probe before the DRS and IFS measurements. During the measurements, the LED 520 is off. As described above, the 2D scanning mirror scans the virtual probe on the tissue and white light camera can monitor the DRS and IFS measurements near-continuously. The length of the imaging probe (from the proximal end to the distal end) is 2 meter. The proximal end splits into four channels (delivery, collection, monitoring, and illumination) as shown in FIG. 10.

The endoscopic QSI system works in conjunction with the therapeutic rhinolaryngofiberscope (Olympus ENF-T3). Although the endoscopic QSI system is capable of performing the endoscopic visual examination it will not replace the ENF-T3 as the main rhinolaryngofiberscope. The 2.2 mm instrument channel of the ENF-T3 can be used for housing the imaging probe which has a diameter of ˜1.5 mm. Its viewing angle is 85 degrees, which allows the physician to see the entire tissue area that is measured by the endoscopic QSI system. Because the ENF-T3 is an approved medical device suitable for the larynx and it is straightforward to put imaging probe through the ENF-T3 instrumentation channel, this implementation of QSI endoscopy should be clinically feasible. There are two modes of operation: normal endoscopy mode and QSI endoscopy mode. In the normal endoscopy mode, the physician can use the ENF-T3 to conduct visual examination of the larynx. The endoscopic QSI system is not used in this mode. During the visual examination, the physician will identify the suspicious tissue area for QSI endoscopic examination. In the QSI endoscopy mode following the visual examination, the QSI measurements can be performed in the following steps:

Positioning the imaging probe: The imaging probe will be threaded onto the distal end of the ENF-T3. The endoscopic QSI system can monitor the whole threading process using its white light monitoring feature. Therefore, the endoscopic QSI system can be used to see when the end of the imaging probe is approaching the tissue. By optimizing the sharpness of the tissue image observed via the monitoring fiberscope, the imaging probe can be properly positioned 5±0.2 mm away from the tissue because its depth of field is 0.2 mm.

DRS measurement: During the DRS measurement, all the light sources except the illumination for the virtual probe are turned off. Similar to what was described above, 400-700 nm broadband light from the light source (Xe lamp, CW 75W Simplicity series, Newport Corp.) is coupled into the delivery single-core fiber (0.22 NA, 200 μm core, Thorlabs M25L01). The 200 μm light spot on the other end of the fiber is imaged to a 80 μm diameter light spot on the proximal end of the delivery fiberscope by using two UV/VIS objective lenses (Olympus UPLSAPO 10× with 0.4 NA and UPLSAPO 4× with 0.16 NA). The delivery fiberscope relays this delivery spot unto the tissue surface with a magnification of 12.5. Consequently, the delivery for the virtual probe has a diameter of ˜1 mm. The tissue reflectance from the same spot is relayed by the collection fiberscope with a demagnification of 12.5 to a light spot (with a diameter of 80 μm) on the proximal end (of the collection fiberscope) which is then coupled into the collection single fiber (0.22 NA, 200 μm core, Thorlabs M25L01) using two UV/VIS objective lenses (Olympus UPLSAPO 10× with 0.4 NA and UPLSAPO 4× with 0.16 NA). Finally, the tissue reflectance is coupled into the spectrometer which consists of a spectrograph (SP150, Princeton Instruments) and monochrome CCD (PhotonMAX, Princeton Instruments). The diffuse reflectance spectrum is then recorded. The pair of spots, delivery and collection, on the tissue surface form the virtual probe where the tissue is interrogated by quantitative spectroscopy. By changing the angle of the 2D scanning mirror, both the delivery and collection are scanned concurrently on the proximal ends of the delivery and collection fiberscopes, respectively. As a result, the virtual probe is scanned over the tissue surface to cover a wide area (with diameter up to ˜6 mm) to perform DRS measurements. The whole scanning spans 0.3 second in 100 steps. One DRS spectrum with 3 ms exposure time will be taken for each step. In turn, 100 DRS spectra are taken from the scanned tissue area in 0.3 second. Because the time span is so short, the motion artifacts are much less compared to the ones for the free-space OSI system. Furthermore, the monitoring white-light camera is recording a movie with a 50 ms exposure time per frame and a frame rate of 20 per second during the whole DRS measurement. Therefore, the recorded video has the full record of the data acquisition and can locate the positions of where all the spectra are taken. The spiral scanning of the diagnostic spot, the DRS exposures and the white-light movie recording are initiated with a single trigger sent out by a DAQ card (National Instruments PCI-6221 M SERIES).

IFS measurement: The IFS measurement will start right after the DRS measurement to minimize the tissue area shift between two measurements. The IFS and DRS measurements are almost identical except for the light source. A UV laser is used (DPSS Lasers Inc., Model 3510-100, 355 nm, 1.0 W at 100 kHz rep rate), instead of the Xe lamp, for excitation. The collection efficiency of the endoscopic QSI system is 12.5 times of that of the free-space QSI system in our DRS signal comparison. Therefore, an UV excitation of 2 μJ energy per diagnostic spot in the endoscopic QSI system is appropriate because 20 μJ UV energy was used per diagnostic spot in the free-space QSI system. The UV laser power is 1 W which means the IFS signal size should be adequate if exposure time is used longer than 2 ms. Based on this estimate, same scanning speed (100-step spiral scanning with 3 ms per step) and CCD exposure time (3 ms) can be used for IFS measurement. Like in the DRS measurement, the white-light monitoring camera is continuously recording the video during the IFS measurement.

The endoscopic QSI system's spectroscopy, imaging, and diagnostic functions can be calibrated tissue phantoms and ex vivo tissues in the same way that the free-space QSI system's functions. Tissue phantoms can be used to measure the instrument's field uniformity, repeatability, accuracy, and defocus tolerance. Ex vivo tissues can be used to measure the instrument's ability to see meaningful contrast, such as the boundary between cancerous and non-cancerous tissue in a variety of organs.

Tissue phantoms can be controlled liquid samples made from mixtures of intralipid, water, hemoglobin, and furan. Intralipid diluted in water is a scattering medium with scattering properties similar to those of tissue. Tissue absorption and fluorescence can be simulated by dissolving hemoglobin and furan in the intralipid solution. Homogeneous phantoms will assess the instrument's field uniformity (the ability to yield identical spectroscopy parameters from identical tissue sections located in different regions of the instrument's field of view); repeatability (by measuring the same phantom multiple times); defocus tolerance (by determining parameters values as a function of defocus), and Instrument accuracy (by comparing the extracted parameters values to those of the set of phantoms with scattering, absorption, and fluorescence properties that span the physiological range).

The homogeneous phantom to test field uniformity, repeatability, and defocus tolerance will consist of a 1:9 10% intralipid to water volume ratio, 1.0 mg/mL of hemoglobin, and 0.5 μg/mL of furan. These concentrations will result in a reduced scattering coefficient, absorption coefficient, and fluorescence emission intensity typical of that found in tissue. The phantom will be positioned precisely 5 mm from the distal tip lens of the endoscope. Ten 6 mm diameter scans are then acquired. Reflectance and fluorescence spectra can be processed using the methods of Zonios and Müller to yield spectroscopy parameters. Using the results of one scan and assuming the phantom is homogeneous over the scan area, any spatial variations in parameter values can be attributed to instrument non-uniformity. If the variations are small, they can be used as a limit for minimum parameter variation that can be resolved by QSI endoscopy. If the variations are large, the results of this measurement can be used to normalize the field. Using the parameter maps from all ten scans and assuming the sample does not change during the duration of the experiment (less than 1 minute), any variations from scan to scan will be due to non-repeatability of the instrument. Since the endoscopic QSI system is a computer controlled instrument, its measurements are highly repeatable.

The endoscopic QSI system has a working distance of 5 mm. To test the defocus tolerance, the phantom used to test uniformity and repeatability will be positioned at various distances from the endoscope tip ranging from 3 mm to 7 mm. For each scan, spectroscopy parameters will be extracted using the appropriate geometry parameter and averaged over the entire 6 mm diameter field. The results will provide information on the suitability of the geometry adjustment and reveal the impact of defocus on spectroscopy parameters measured by the endoscopic QSI system. During tissue measurements when the sample—instrument separation is unknown, r_{c}′ becomes an optimization parameter, instead of a constant, in Zonios' model.

Define accuracy as the ability of an instrument to correctly determine the scattering, absorption, and fluorescence properties of a sample. To assess QSIE's accuracy, two sets of phantoms can be used, one for reflectance and another for fluorescence. For reflectance spectroscopy, a set of phantoms spanning physiologically relevant reduced scattering and absorption coefficient ranges (0.7 mm⁻¹¹<μd s′<3.3 mm⁻¹, 0 mm⁻¹<μₐ<2.0 mm⁻¹) can be prepared from intralipid and hemoglobin. The reduced scattering coefficient range corresponds to an intralipid dilution range of approximately 1:20<dilution<1:5, where the dilution is of standard 10% intralipid in water. To assess fluorescence capabilities, a set of phantoms with 1:9 volume ratio 10% intralipid and water, 1.0 mg/mL hemoglobin, and furan concentrations of 0.25, 0.5, and 0.75 μg/mL will be created. This range is physiologically relevant and can be used to assess the free-space QSI instrument. Spectroscopy data can be acquired from each phantom over a 6 mm diameter area. The spectra can be modeled by the methods of Zonios and Müller to yield spectroscopy parameters. Accuracy of scattering, absorption, and fluorescence measurements will be determined by comparing outputted scattering, absorption, and fluorescence parameters to input parameters. For scattering, the expected reduced scattering coefficient for a given intralipid concentration can be determined. For absorption, the amount of hemoglobin powder mixed with intralipid will determine the hemoglobin concentration. For fluorescence, a phantom with twice the actual furan concentration of another phantom can yield extracted furan concentration parameter twice that of the other phantom.

Tissue phantoms cannot mimic all of the scattering, absorption and fluorescence properties of tissue. The most important aspect of any cancer imaging system is the ability to see the contrast between diseased and healthy tissue. For the endoscopic QSI system, the ability to see contrast can be verified with ex vivo tissue. Conducting experiments with tissue ex vivo is considerably less difficult than in vivo data acquisition and the morphology and biochemistry of the epithelium has not been significantly altered. The tissue preparation and handling facilities in the laboratory are well suited for ex vivo experiments. The endoscopic QSI system can scan excised tissue from colon, esophagus, and larynx as these are all organs typically accessed by endoscopes.

A specimen can be placed 5 mm from the distal tip of the endoscope and spectroscopy will be conducted over a 6 mm diameter circle. Parameter maps are extracted from the spectra using the methods of Zonios and Müller. The specimen can be fixed in formalin and processed by histopathological analysis which is similar to that used in the ex vivo tissue. Spectroscopy parameter maps can be compared to various pathological parameter maps with emphasis on seeing contrast between cancerous and healthy tissue.

The endoscopic QSI system has field uniformity, repeatability, and accuracy comparable to those of the free-space QSI system. Defocus can have minimal effect on the accuracy of extracted spectroscopy parameters because the model compensates for illumination spot enlargement. However, since defocus leads to a larger illumination spot, there will be overlap between data measured from neighboring tissue points. The ex vivo tissue measurements demonstrate that QSI can detect the contrast between cancerous and non-cancerous tissue ex vivo, although different tissue types can result in different spectroscopy parameters showing contrast. As a result, there is not one universal diagnostic algorithm for all organs.

Cancers of the larynx and vocal cords are difficult to detect since the locations of these organs are not as accessible as the rest of the oral cavity. The current standard of care uses laryngoscopes or a Hopkins rod-lens telescope to visualize the throat. There are now commercial laryngoscopes capable of video rate imaging (Olympus). The endoscopic QSI system can improve diagnosis of laryngeal and vocal cord cancers by giving a quantitative results and potentially reducing the number of biopsies. The latter will be particularly important in this region of the body because cutting or disrupting the vocal cords can lead to changes in voice and in severe cases, loss of speech. In instances when taking a biopsy is highly undesirable, for example if the patient relies on his/her voice to earn a living, spectroscopy can serve as the final diagnosis. For these reasons, the larynx and vocal cords serve as an important application for the endoscopic QSI system.

The surgeon uses a rhinolaryngofiberscope to visualize the throat. Only local anesthesia is required for larygnoscopy, so the patient can be conscious during the entire procedure. Once the surgeon has identified the suspicious region, the endoscopic QSI system imaging probe will be threaded into the 2.2 mm diameter instrument channel of the laryngoscope. For each suspicious site, a 6 mm in diameter diagnostic area will be examined by spectroscopy, yielding scattering, absorption and fluorescence parameter maps. In addition, the contralateral uninvolved tissue can be imaged as normal control for each patient. Each spectroscopy measurement can require about 0.6 seconds for data acquisition, and spectroscopy adds no more than five minutes to the regular procedure time. After spectroscopy measurements are complete, the endoscope will be removed and if necessary, biopsy forceps will be inserted into the working channel and necessary biopsies taken.

Spectra measured from the patients can be processed using the models of Zonios and Müller or other models to yield parameter maps. The parameter maps compared to pathology results are considered, which is the current clinical standard. From these patients, a combination of parameters is used that enable spectroscopy to distinguish between normal and abnormal tissues.

DRS and IFS parameter maps work together to provide quantitative tissue information. Using DRS, the diffuse reflectance spectrum is analyzed to extract structural/morphological properties such as hemoglobin concentration, oxygen saturation, and average diameter and density of scatterers. With IFS, the contributions from different fluorophores (e.g. NADH and collagen) can be obtained. Furthermore, the sampling volume for DRS is thicker than that of IFS because the white-light illumination (used for DRS) has deeper penetration than the UV excitation (used for IFS). This depth difference in sampling volume may provide an opportunity to extract parameters in 3D fashion. The movies records by the white-light camera are used to overlap the DRS and IFS parameter maps. Therefore, the complementary tissue information provided by DRS and IFS can be combined to improve the specificity of the spectral diagnosis. To correlate spectroscopic imaging results to histopathological grading of biopsies, the surgeon will indicate the tissue site to be biopsied on the white light image presented by the endoscopic QSI system. Since pixels on the white light image are correlated to spectroscopic measurements, this allows us to determine which measurements came from the tissue site biopsied. Each histopathological grading of biopsy can be correlated to DRS and IFS parameters. Therefore, all DRS and IFS parameters can be grouped according to disease states. In addition, data from clinically normal sites can be included as normal/non-diseased based on clinical diagnosis only. Bayes' theorem, can be used to develop a classification algorithm to test the ability of the endoscopic QSI system for distinguishing between abnormal and normal tissues, as well as dysplastic from non-dysplastic tissues.

While the present invention has been described herein in conjunction with a preferred embodiment, a person with ordinary skill in the art, after reading the foregoing specification, can effect changes, substitutions of equivalents and other types of alterations to the system and method that are set forth herein. Each embodiment described above can also have included or incorporated therewith such variations as disclosed in regard to any or all of the other embodiments. Thus, it is intended that protection granted by Letters Patent hereon be limited in breadth only be definitions contained in the appended claims and any equivalents thereof.

1. A system for quantitative spectroscopic imaging of tissue comprising:
a light source system;
a scanning system that scans a light region across a tissue surface; and
a detector system that detects light from the scanning light region tissue surface.

2. The system of claim 1 further comprising a processor that provides an image of the tissue.

3. The system of claim 1 wherein the light region comprises a light spot.

4. The system of claim 1 wherein the light region comprises an illumination region and a light collection region.

5. The system of claim 1 wherein the scanning system comprises a moveable mirror.

6. The system of claim 1 wherein the scanning system optically couples the light source to a proximal end of endoscopic probe.

7. The system of claim 1 wherein the scanning system optically couples a proximal end of the endoscopic probe to the detector system.

8. The system of claim 1 wherein the endoscope probe comprises a plurality of optical fibers.

9. The system of claim 1 further comprising a spectrograph.

10. A method for quantitative spectroscopic imaging of tissue comprising:
scanning a light region across a tissue surface; and
detecting light from the scanning light region on the tissue surface to form an image.

11. The method of claim 10 further comprising processing collected an image of the tissue.

12. The method of claim 10 further comprising scanning a light spot across a tissue surface.

13. The method of claim 10 wherein the light region comprises an illumination region overlapping a light collection region.

14. The method of claim 10 further comprising operating a moveable mirror to scan the light region across the tissue surface.

15. The method of claim 10 further comprising optically coupling the light source to a proximal end of endoscopic probe.

16. The method of claim 10 further comprising using a fiber optic probe to deliver light from a light source onto the tissue surface.

17. The method of claim 16 further comprising coupling a laser light source and a broadband light source to a proximal end of the fiber optic probe.

18. The method of claim 10 further comprising detecting a video image of the tissue with a camera.

19. The method of claim 10 further comprising detecting fluorescence from the tissue with a detector.

20. The method of claim 19 further comprising processing the detected fluorescence to generate intrinsic fluorescence data.
